(19) United States
(12) Patent Application Publication
(10) Pub. No.: US 2024/0335216 A1
(43) Pub. Date: Oct. 10, 2024

(54) PUBIC SYMPHYSIS BONE PLATE AND SET

(71) Applicant: OT MEDIZINTECHNIK GMBH, München (DE)

(72) Inventors: Ulrich SCHREIBER, München (DE); Wilhelm FRIEDL, Haibach (DE)

(21) Appl. No.: 18/579,478

(22) PCT Filed: Jul. 13, 2022
(86) PCT No.: PCT/EP2022/069589

§ 371 Date: Jan. 15, 2024

(30) Foreign Application Priority Data
Jul. 13, 2021 (DE) 10 2021 118 087.2

(51) Int. Cl. A61B 17/80; A61B 17/68

(52) U.S. Cl. CPC A61B 17/8014; A61B 17/8057; A61B 2017/681

(57) ABSTRACT

The present invention relates to a pubic symphysis bone plate (100) for the surgical treatment of damages, in particular fractures or breaks, of the pelvis in the region of the symphysis, anterior pelvic ring up to and including the acetabulum in the case of suitable fractures. The present invention further relates to a set comprising a pubic symphysis bone plate (100) according to the present invention and further comprising at least one cannulated screw or creeping screw (120), preferably made of titanium alloy, preferably anodized, at least one interlocking screw (130) and/or a target instrument.

### CROSS-REFERENCE TO RELATED APPLICATION

This application is a U.S. national stage entry of international application no. PCT/EP2022/069589 filed on Jul. 13, 2022, and claims priority to and the benefit of German application no. DE 10 2021 118 087.2 filed on Jul. 13, 2021, the entire contents of which are incorporated by reference herein.

### FIELD OF THE INVENTION

The present invention relates to a pubic symphysis bone plate and a set comprising a pubic symphysis bone plate.

### BACKGROUND OF THE INVENTION

The pubic symphysis (symphysis pubica) is a ventrally located bone joint of the pelvic ring of mammals and humans, at which the right and left bony halves of the pelvis, more precisely the pubic bones (os pubica) of both sides, are connected at the front. The two halves of the pelvis are connected by fibrous cartilage (symphysis). The bones adjacent to the pubic symphysis are referred to as symphysis bones.

In the treatment of pelvic fractures, particularly unstable pelvic ring fractures (such as those described as type B or C fractures in the AO (Osteosynthesis Working Group (https://aotrauma.aofoundation.org/))) and pelvic ring fractures following low-energy trauma in osteoporosis (according to Rommens/Hofmann: FFP fractures type III and IV), screw or plate osteosynthesis (bone connections) may be used for stabilization.

### SUMMARY OF THE INVENTION

One object of the present invention is to specify a further bone plate which preferably enables a less invasive and more stable fixation, in particular a pubic symphysis bone plate.

Furthermore, a set is to be specified.

The object according to the present invention may be achieved by a pubic symphysis bone plate with the features as described herein and by a set with the features as described herein.

The present invention relates to a pubic symphysis bone plate for the surgical treatment/stabilization of damages, in particular fractures or breaks of the pelvis in the region of the symphysis or the anterior pelvic ring up to the acetabulum on both sides. The pubic symphysis bone plate comprises a plurality of through-openings.

The through-openings preferably extend through the lesser extent or the thickness or strength of the pubic symphysis bone plate, i.e. e.g. at an angle not equal to 0°, for example between 30° and 150°, e.g. 90°, to its main extension plane, e.g. the top view or front view or largest surface. In use, the direction of the through-openings may be referred to, e.g., as ventral-dorsal (or vice versa).

The present invention further relates to a set comprising a pubic symphysis bone plate according to the present invention and furthermore to at least one cannulated screw or creeping screw, at least one interlocking screw and/or at least one connecting screw for the releasable connection of a target instrument and/or a target instrument.

The bone screw/creeping screw is used to interlock the pubic symphysis bone plate in the upper pubic ramus. It is preferably inserted polyaxially for this purpose.

Due to the polyaxial screw mechanism, the bone screw/creeping screw may be inserted, deviating from the through-opening axis, in a cone of up to 20° or more. The bone screw/creeping screw may be blocked in the bone plate at a stable angle and thus fix the bone fragments in their position.

The creeping screw is preferably made of a titanium alloy, steel or other material or a combination thereof, preferably anodized with a surface finish against corrosion, i.e. provided with a firmly adhering oxide layer against corrosion and abrasion.

The cannulation extends along the longitudinal extent of the creeping screw in its interior and comprises an internal diameter for example between 1.5 mm and 3 mm.

The interlocking screw(s), on the other hand, serve(s) to interlock the pubic symphysis bone plate in the medial area of the symphysis. They may be used individually or in groups.

The interlocking screw is preferably angularly stable and may preferably be connected to the bone plate in a polyaxial or monoaxial manner.

The target instrument is described in more detail below.

Embodiments according to the present invention may comprise some, several or all of the features mentioned below in any combination, unless a specific combination is recognized by the person skilled in the art as being technically impossible. Advantageous developments of the present invention are each also subject-matter of the embodiments.

In all of the following statements, the use of the expression “may be” or “may have” and so on, is to be understood synonymously with “preferably is” or “preferably has,” and so on respectively, and is intended to illustrate embodiments according to the present invention.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”. This understanding is also equally encompassed by the present invention as the interpretation that a numerical word, for example, “one” (or “a/an”) may alternatively mean “exactly one”, wherever this is evidently technically possible in the view of the person skilled in the art. Both of these understandings are encompassed by the present invention and apply herein to all used numerical words.

Advantageous developments of the present invention are each also subject-matter of embodiments.

Whenever an embodiment is mentioned herein, it is then an exemplary embodiment according to the present invention.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

When the term “angular stability” is used herein, it may be understood to mean a condition in which the angle set between e.g. the screw and the plate is maintained by a force, torque or a combination thereof acting on the screw. The angular stability may be generated monoaxially, i.e. at a single angle, or polyaxially at different angles.

In some embodiments of the pubic symphysis bone plate according to the present invention, at least one of the through-openings comprises at least two diameters differing from each other and/or a step within its inner lumen, in others not.

In several embodiments, at least one of the through-openings comprises an internal thread. In some embodiments, at least one of the through-openings comprises no internal thread, in other embodiments this is not the case.

In some embodiments of the pubic symphysis bone plate, an upper edge of the pubic symphysis bone plate in use comprises a middle section which extends higher in a caudal-cranial direction in use than sections bounding it left and right.

This may improve orientation during plate positioning.

In several embodiments, the pubic symphysis bone plate comprises a recess on its front side (ventral in use). The recess is designed to receive at least a section of the depth or thickness of a compression plate. The compression plate or embodiments thereof are described in more detail below.

In some embodiments, the recess of the pubic symphysis bone plate and the side of the compression plate facing the pubic symphysis bone plate may have geometries that correspond to each other or that connect them to each other in a form-fit manner, e.g., be designed both circular, both elliptical, both rectangular or a combination of geometric figures (rectangular in a first section and e.g. circular in a second section, or the like), etc. However, the form-fit connection may also be limited to only one spatial direction. In this case, the recess and the compression plate could have the same extension in one direction, e.g. the same width, or the same length, etc.

In several embodiments, the pubic symphysis bone plate comprises at least one opening having an internal thread which is provided and suitable for screwing in a compression screw for screwing a compression plate to the pubic symphysis bone plate.

In some embodiments of the set according to the present invention, at least one of the creeping screws comprises a head end and a free end lying opposite the head end.

The creeping screw comprises a first external thread in the region of its head end and a second external thread in the region of its free end, wherein the first external thread has a larger or equal external diameter than/as the second external thread.

The “head end region” herein may in some embodiments be the head and/or the end itself, in others it is a section adjoining the head, in certain embodiments it encompasses both of the foregoing options, as head/end and adjoining section.

In several embodiments, a thread-free section is arranged on the creeping screw between the first external thread and the second external thread, at least on its outer side.

In some embodiments, the head end or a section close to the head of the at least one creeping screw comprises a section which has an outer diameter which is larger than the outer diameter of a section of the free end, which in turn lies opposite the head end. Alternatively or additionally, the section at the head end or the section that is closer to the head end than the free end of the creeping screw comprises a larger outer diameter than the thread-free section.

The section with the larger outer diameter serves in particular to support the creeping screw on or in the bone.

In some embodiments, the creeping screw comprises a first and a second external thread, wherein the first and second external threads differ with regard to at least one property, for example in the pitch. For example, a double thread pitch may be provided for faster screwing into the bone.

In some embodiments, the creeping screw may comprise a threaded section or a thread on the head (e.g. on its outer side) or on the head region (e.g. on its outer side) itself, which, when screwed into the pubic symphysis bone plate, combines, e.g., with the internal thread of the through-hole in the pubic symphysis bone plate in order to form an angularly stable connection. In such embodiments, the threaded section on the head may correspond to the first external thread or be designated as such.

In several embodiments of the set, at least one of the interlocking screws comprises a head end and a free end lying opposite the head end.

The interlocking screw has a third external thread in the region of its head end and a fourth external thread in the region of its free end, wherein the third external thread has a larger diameter or outer diameter than the fourth external thread.

In some embodiments, the interlocking screw comprises a third and a fourth external thread, wherein the third and fourth external threads differ with respect to at least one property, for example in the pitch.

In several embodiments, the external thread in the region of the head end comprises a conical and/or metric thread, whereas in certain embodiments, the external thread in the region of the free end comprises a thread geometry optimized for fixing it in the cancellous and/or cortical bone.

The head or the head end itself may e.g. comprise a conical and/or metric thread so that the head may form a releasable connection with the bone plate and thus create angular stability and a protection against “migration” in the axial direction towards the head end.

In some embodiments, any one of the threads or external threads mentioned herein may be a thread with a fixed diameter or a thread with a tapered diameter or a diameter that tapers along the longitudinal axis of the screw. This may apply in particular to the creeping screws mentioned herein, optionally also to the interlocking screws mentioned herein.

In several embodiments, the target instrument optionally comprises respectively a target bow, a polyaxial guiding device, also referred to herein as a “joystick”, and/or a guiding device with monoaxial openings, also referred to herein as a “guiding block”.

In some embodiments, the target instrument comprises a plate holder configured to be releasably connectable to the pubic symphysis bone plate.

In certain embodiments, all components of the set are provided to be releasably connectable to each other. Devices for the corresponding releasable connection are provided.

In several embodiments, the plate holder comprises at least one through-opening for passing or advancing a K-wire therethrough, in others it does not.

In certain embodiments, the target bow comprises at least one through-opening for passing a K-wire therethrough, in other embodiments it does not.

In several embodiments, the compression plate is releasably connected to the bone plate.

In some embodiments, the bone screws are prevented from unscrewing axially (towards the screw head of, e.g., a compression plate or the like) by covering their heads.

In several embodiments, at least one bone screw is prevented from rotating (angular stability) by a frictional connection and/or form-fit connection.

In some embodiments, the set comprises a compression plate and a compression screw for screwing the compression plate against a surface of the pubic symphysis bone plate. Preferably, the compression plate is screwed into the recess to receive at least a section of the depth of a compression plate.

In several embodiments, the compression plate forms a form-fit connection with the bone plate.

In several embodiments, the set has a positioning handle, which is prepared in particular for the releasable connection to the target bow. For this purpose, target bow and positioning handle may have corresponding tongue-and-groove systems, pin-opening systems, key-lock systems or the like. The positioning handle may be used by the surgeon to maneuver the set or the target bow in the surgical area and may then be separated from the target bow.

In some embodiments, the tongue-and-groove systems, pin-opening systems, key-lock systems or the like on the part of the target bow, which serve for releasable connection to the positioning handle, are further provided for permanently or releasably connecting further parts of the target instrument to the target bow, in particular the guiding devices (joystick and guiding block).

In several embodiments, the positioning handle comprises one or several openings for receiving a K-wire.

In some embodiments, the pubic symphysis bone plate is made of materials other than titanium, such as polyether ether ketone (PEEK), stainless steel or carbon fiber composite or magnesium or other resorbable material, or it comprises at least one other material or combinations thereof. These materials may optionally be used, or will be used, for any or all of the components described herein (creeping screws, plate, compression plate, compression screw, interlocking screws), either as the sole material or as part of the component.

In several embodiments, the creeping screw comprises a polyaxiality of ±10° (0° to 20°) up to ±20° or more relative to the center axis of the respective borehole.

Structurally, in some embodiments, the bone plate comprises one or a plurality of through-openings which enable at least one axial alignment of the bone screw.

The longitudinal axis of the bone screw may be pivoted polyaxially around the axial preferred direction and with the pivot point within or close to the bone plate in a conical angle range discretely or continuously of up to 5°, 10°, 15°, 20° or more or in a different angle range. This may make it possible to position the creeping screw such that it can be guided (on both sides) precisely through the narrowest point of the upper pubic ramus and past the acetabulum (hip joint socket) without touching it.

In some embodiments, the interlocking screw comprises a shaft diameter of Ø 3, 5, 7 mm or another diameter.

In several embodiments, the interlocking screws block themselves.

In some embodiments, the blocking takes place between the bone screw, interlocking screw and/or creeping screw, and the bone plate.

In some embodiments, the target instrument and/or some or all of its individual components and/or some or all parts of the individual components, optionally each in any combination, are, preferably predominantly, radiolucent.

In several embodiments, the target instrument and/or its individual components or parts of the individual components may be made of plastic, for example carbon fiber reinforced PEEK (CFRP), PEEK, POM, etc., anodized aluminum, anodized titanium alloy, and/or stainless steel, or may comprise any technically possible combination of these materials.

In some embodiments, the pubic symphysis bone plate comprises an anatomically preformed, curved design.

In several embodiments, the pubic symphysis bone plate is symmetrical.

In some embodiments, the pubic symphysis bone plate comprises an opening angle that matches a female pelvis, in others an opening angle that matches a male pelvis.

In several embodiments, the creeping screws are interlocked individually, in others together.

When it is referred to “blocking” herein, this particularly encompasses fixing the screw(s) in a desired angular position, in particular relative to the plate or another screw. Blocking may also preferably secure against so-called backing-out, i.e. against unwanted loosening of the screw against the direction along which it was screwed in.

In some embodiments, the pubic symphysis bone plate comprises at least one connection point, for example a blind opening, a through-opening or the like, for connecting it, preferably releasably, to a section of the target instrument, in particular for connecting it to a section of the plate holder.

In several embodiments, the pubic symphysis bone plate comprises through-openings, for example bores, for receiving K-wires, in particular for temporarily fixing the pubic symphysis bone plate to the pelvic bone. These through-openings may have a diameter of 1.6 mm to 2.5 mm, preferably a diameter of 2 mm.

In some embodiments, the middle section of the upper edge serves as an orientation for the surgeon with regard to the implantation height in the cranial direction in that it is designed such that it is flush with the upper edge of the pubic bone during implantation, which may be checked visually, by palpation or by fluoroscopy.

In some embodiments, the creeping screws have a full thread, in others they do not.

Preferably, the creeping screws have a receptacle for a screwdriver at the front side of their head end.

In some embodiments, the creeping screws and/or interlocking screws are self-tapping and/or self-drilling.

In several embodiments, the creeping screws have different lengths, preferably between 40 mm and 200 mm.

In some embodiments, the creeping screws comprise a cannulation with an inner diameter of 1 mm and up to 3.5 mm, preferably a diameter of 3.2 mm.

In several embodiments, the first external thread of the creeping screw serves to provide bony support for the creeping screw in the pelvic bone. It then has a larger or the same diameter as the thread-free part or the second external thread. In this way, support may also be provided, for example by self-drilling or self-tapping external threads.

In some embodiments, the creeping screw comprises a widened outer diameter instead of a first outer thread at its head end near the head. This may also serve for bony support.

In several embodiments, the compression plate is a two-part component comprising a plate and a screw, which is preferably pre-assembled.

In some embodiments, the compression plate is made of or comprises titanium, in others it is made of or comprises steel, in others it is made of or comprises PEEK, in others it is made of or comprises a CFRP. Combinations of these materials are also encompassed by the present invention.

In several embodiments, there is a small amount of “play” between the compression plate and the creeping screw. In these embodiments, the compression plate only protects against backing-out.

In some embodiments, the compression plate presses on the creeping screw such that there is a frictional connection, i.e. no “play”. In these embodiments, the compression plate not only protects against backing-out, but also ensures that the creeping screw is blocked at a stable angle.

In several embodiments, the plate holder of the target instrument may be designed to be universal, i.e. suitable for different bone plates. This means that it may be used, for example, also in connection with a humerus plate.

In some embodiments, the target instrument comprises one or more radiopaque markers (e.g. integrated) for visualizing the beam path. The marker may be, or have, e.g., a pin and/or a circular geometry which (pin) appears as a point (not a line) in the image converter in the case of a parallel beam path, or (circle) which then appears as a true circle (not an oval) in the case of a parallel beam path.

An insertion option for one or several such markers, e.g. pins, may be provided, which may give visual feedback on the alignment to the beam path or the alignment of the target instrument.

Such designs facilitate implantation and the desired positioning of the bone plate with respect to the anatomy and, thus, also the positioning/alignment of the bone screws/creeping screws.

In several embodiments, the pubic symphysis bone plate has an anatomical shape. In some embodiments, it has a ventral side that is at least partially, or substantially, convex in use, and a dorsal side that is at least partially or substantially concave in use.

In several embodiments, the pubic symphysis bone plate has a cranial edge (regarding its use) and a caudal edge. The cranial edge may be convex or have a convex edge section, the caudal edge may also be convex or have a convex edge section.

In some embodiments, the pubic symphysis bone plate comprises, in a caudal-cranial direction, at least in sections, a bi-convex shape.

In some embodiments, the pubic symphysis bone plate is in particular not a flat structure, and/or not all entry or introducing surfaces of the through-openings of the pubic symphysis bone plate lie in the same plane.

In some embodiments, the pubic symphysis bone plate comprises through-openings in more than one row in a caudal-cranial direction. Thus, a, or several, cut through the pubic symphysis bone plate lying in the frontal plane or implanted on the standing patient, lying in the sagittal plane may extend through more than just one through-opening in a caudal-cranial direction.

In some embodiments, the compression plate is not annular, not shaped like a nut and/or not shaped like a bushing.

In several embodiments, the connection between the pubic symphysis bone plate and the bone is done only by screws, or by screws and at least one compression plate, preferably without additional releasable thread-bearing inserts such as bushings, nuts, compression rings, anti-rotation rings and/or the like.

Some or all embodiments according to the present invention may have one, several or all of the advantages mentioned above and/or below.

One advantage of the present invention may be that the pubic symphysis bone plate according to the present invention is implanted frontally (ventrodorsally) on the pubic symphysis of the human pelvis. Here, it may advantageously contribute to the stabilization of the anterior pelvic ring in the case of an existing fracture, for example due to an accident, or in the case of a pathological or osteoporotic fracture, for example in the case of osteoporotic bones. The present invention may advantageously be used to treat a number of different pelvic fractures, regardless of whether they are caused by a high-speed or low-speed trauma.

A further advantage of the present invention may be its high accuracy of fit. Due to the anatomically preformed plate design and the polyaxiality of the creeping screws, it may be easily and precisely matched to female and male pelvises.

The high primary stability of the present invention enables accelerated mobilization of the patient, which is usually accompanied by an improved healing process. This may represent a further advantage.

A further advantage of the present invention may be that the pelvic plate and target instrument allows minimally invasive approaches to stabilize the anterior pelvic ring to the bilateral acetabulum. Due to its intuitive and safe use, it can make the operation easier for the user and, thus, also for the patient.

The blocking of the creeping screws and interlocking screws of the present invention advantageously prevents backing-out.

The angularly stable blocking of the creeping screws and interlocking screws advantageously provides additional protection against relative movements. This may also contribute to a faster healing process and may thus represent a further advantage of the present invention.

### BRIEF DESCRIPTION OF THE DRAWINGS

In the following, the present invention is described purely exemplarily with reference to the accompanying figures. In them, identical reference numerals denote identical or similar components. The following applies:

FIG. 1 shows a pubic symphysis bone plate according to the present invention in a first embodiment in a perspective view of;

FIG. 2 shows the pubic symphysis bone plate of FIG. 1 from the top (cranial-caudal);

FIG. 3a shows the pubic symphysis bone plate in a second embodiment in a perspective view, together with a compression plate;

FIG. 3b shows the compression plate of FIG. 3a from the front/top;

FIG. 4 shows various embodiments of the creeping screw;

FIG. 5 shows an embodiment of an interlocking screw;

FIG. 6a shows a plate holder as part of a target instrument;

FIG. 6b shows a target bow as part of the target instrument;

FIG. 6c shows a guiding block as part of the target instrument;

FIG. 6d shows a joystick as part of the target instrument;

FIG. 6e shows the pubic symphysis bone plate with parts of the target instrument and a positioning handle;

FIG. 7 shows the pubic symphysis bone plate with parts of the target instrument in a common use;

FIG. 8 shows the pubic symphysis bone plate with parts of the target instrument in a further common use;

FIG. 9a shows a final osteosynthesis in a view from the front (anterior-posterior) towards the pubic symphysis bone plate according to the present invention; and

FIG. 9b shows the final osteosynthesis of FIG. 9a with transparent pelvic bones.

### DETAILED DESCRIPTION OF THE INVENTION

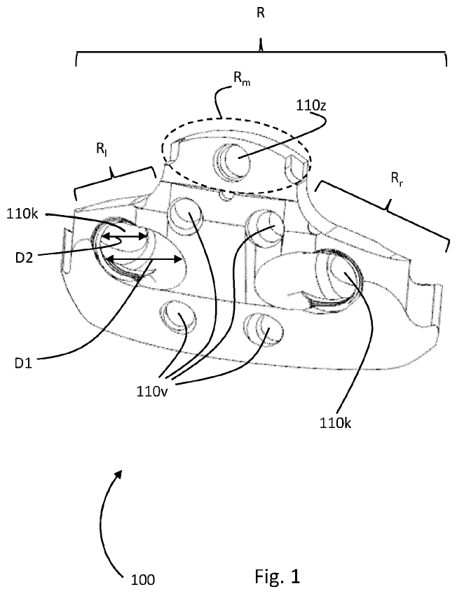

FIG. 1 shows a pubic symphysis bone plate 100 according to the present invention in a first embodiment a perspective view.

The pubic symphysis bone plate 100 serves for the surgical treatment of damages, in particular fractures or breaks, of the pelvis in the area of the symphysis and anterior pelvic ring. It is preferably made of a titanium alloy, preferably anodized, or comprises such an alloy. Alternatively or additionally, the pubic symphysis bone plate 100 may be made of other materials, for example polyether ether ketone (PEEK), stainless steel or carbon fiber reinforced plastics (CFRP) or may comprise at least one other material.

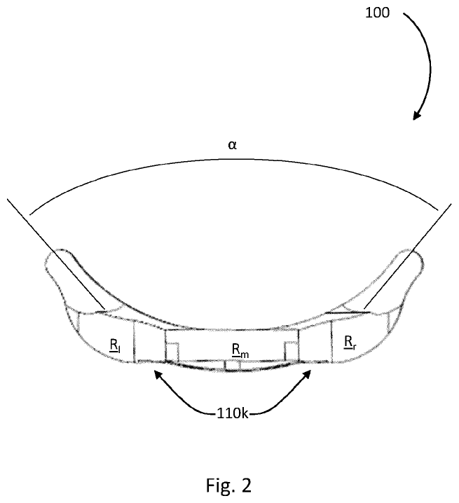

The pubic symphysis bone plate 100 of FIG. 1 optionally comprises an anatomically preformed, e.g., symmetrical (see also FIG. 2) design. In particular, it may match to a female or a male pelvis.

The pubic symphysis bone plate 100 comprises a plurality of through-openings, of which purely exemplarily, through-openings 110k, 110v and 110z, which designate different types of through-openings, are present in the embodiment of FIG. 1.

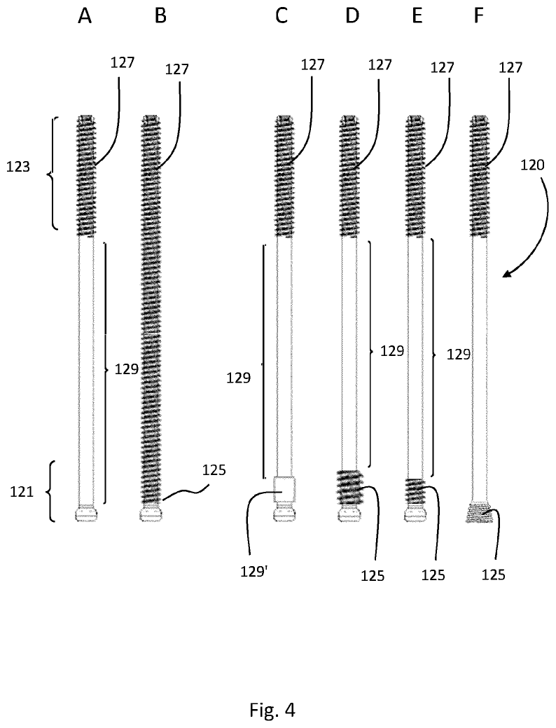

The two through-openings designated by reference numeral 110k, which here optionally comprise at least two different diameters D1, D2 and/or a step within their inner lumen, serve to receive and/or block creeping screws 120, which are not shown here but are described in detail in particular with regard to FIG. 4. Such creeping screws 120 may be received in the through-openings 110k, in particular polyaxially, in particular in an angular range indicated supra.

An optional blocking of creeping screws 120 inserted into the through-openings 110k of the pubic symphysis bone plate 100 ensures angular stability of the creeping screws 120 and provides a protection against the so-called backing-out, the unwanted loosening of the creeping screws 120 against the direction in which they were screwed in.

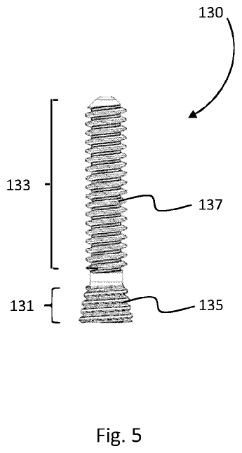

In FIG. 1, through-openings 110v-here exemplarily four-serve to receive and/or block interlocking screws 130, which are not shown here, but are described in detail in particular with regard to FIG. 5. The interlocking screws 130 may be accommodated monoaxially or polyaxially, polyaxially in particular in an angle range of ±5° (0° to 15° or 20° or more). The optional blocking ensures angular stability of the screws and provides protection against backing-out.

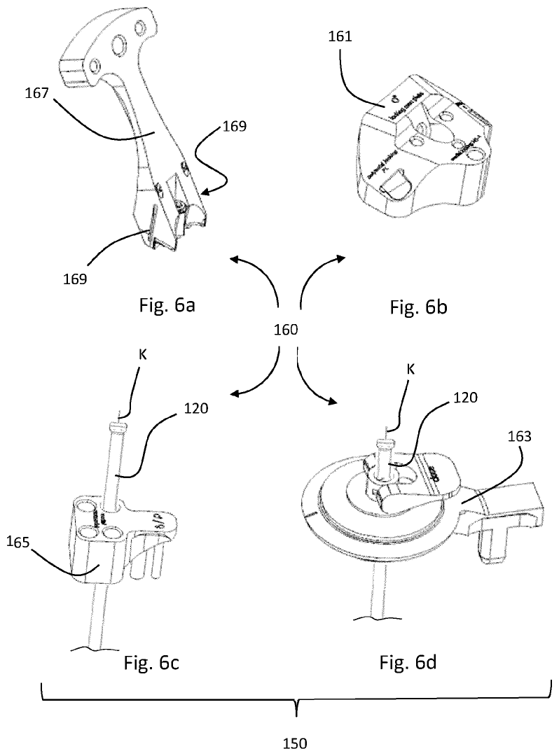

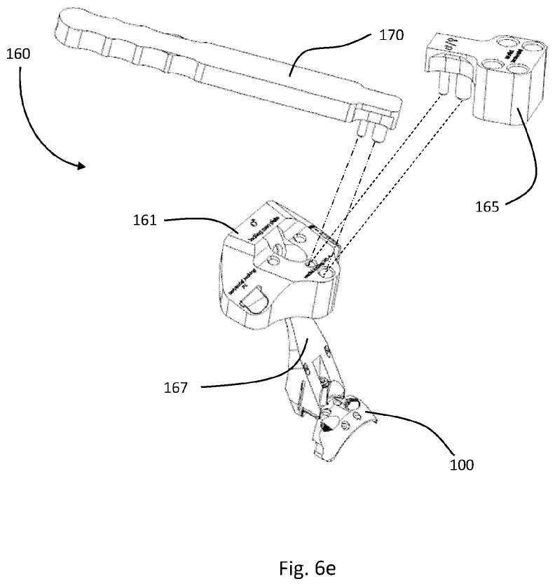

The at least one through-opening 110z serves to receive, in particular releasably, a target instrument 160, which is not shown in FIG. 1, but is described in detail in particular with regard to FIGS. 6, 7 and 8.

The through-opening 110z is optionally provided in a section which lies below a central section Rₘ of an upper edge R, which extends higher in a caudal-cranial direction in use than further sections (Rₗ, Rᵣ) of the edge R bounding it on left and right.

As can be seen in FIG. 1, the, or a, through-opening 100z is thus preferably provided in an upper end region, an apical section of the pubic symphysis bone plate 100 and/or as the highest opening.

Instead of a through-opening 100z for the releasable connection between the pubic symphysis bone plate 100 and the target instrument 160, a plurality of such through-openings 100z may be provided. As an alternative to a second and/or further through-opening 100z, openings may be provided for the same purpose and/or in the same configuration as described herein, which are not through-openings but, for example, blind openings. The through-opening 100z shown in FIG. 1 is also not necessarily a through-opening. Alternatively, it could also be a blind opening.

As an alternative to a second and/or further through-opening 100z, e.g., openings for centering pins, stops, steps, notches as anti-rotation protection and/or the like may be provided.

Optionally, at least one of the through-openings 110k, 110v, 110z of the pubic symphysis bone plate 100 comprises an internal thread, optionally at least one of the through-openings 110k, 110v, 110z has no internal thread.

FIG. 2 shows the pubic symphysis bone plate 100 of FIG. 1 from the top.

Reference is made to the description of FIG. 1.

The optionally anatomically preformed, here exemplarily symmetrical design of the pubic symphysis bone plate 100 may be seen in this view.

By selecting the appropriate opening angle α of the two through-openings 100k, the pubic symphysis bone plate 100 may be, or will be, easily matched to a female or male pelvis by the manufacturer.

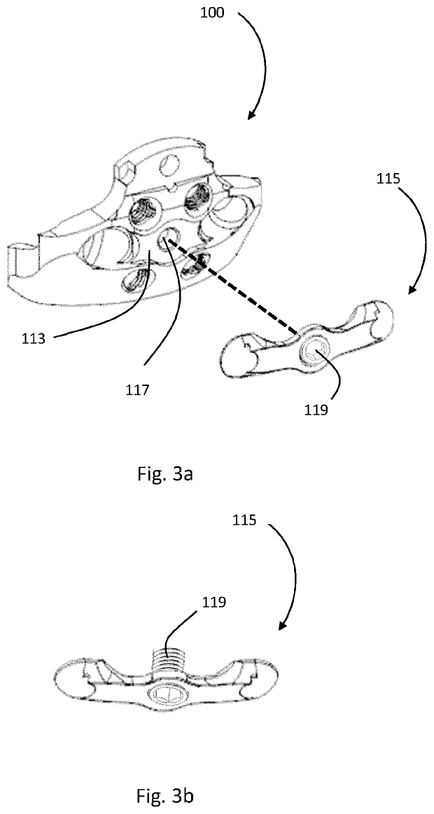

FIG. 3a shows the pubic symphysis bone plate 100 in a second embodiment in a perspective view; a compression plate 115 is also shown.

The pubic symphysis bone plate 100 of FIG. 3a substantially corresponds to the pubic symphysis bone plate 100 of FIG. 1. Therefore, in order to avoid repetition, reference is made to the description thereof. The differences will be discussed hereafter.

The pubic symphysis bone plate 100 comprises an optional recess 113 on its front side for receiving at least part of the depth or thickness of a compression plate 115.

The recess 113 of the pubic symphysis bone plate 100 and the side of the compression plate 115 facing the pubic symphysis bone plate 100, or the edge of the recess 113 and the edge of the compression plate 115, may have corresponding geometries or geometries that positively connect to each other, at least in sections after connecting the compression plate 115 to the pubic symphysis bone plate 100.

In use, the compression plate 115 serves to block the creeping screws 120 (not shown in FIG. 3a) or to prevent them from loosening in the sense of backing-out, with the features and advantages mentioned herein.

The pubic symphysis bone plate 100 comprises at least one further opening 117 for passing through a compression screw 119. Preferably, the opening 117 comprises an internal thread for screwing in the compression screw 119.

FIG. 3b shows the compression plate 115 of FIG. 3a from the front/top. Reference is made to the description of FIG. 3a.

The compression plate 115 may be made of titanium or PEEK. It is optionally presented as a two-part component consisting of a plate and compression screw 119, which may preferably be pre-assembled before use.

FIG. 4 shows various embodiments of creeping screws 120 (A to F), which may be encompassed by the pubic symphysis bone plate 100 according to the present invention or by the set according to the present invention. The creeping screws 120 serve to interlock the pubic symphysis bone plate 100 in the upper pubic ramus (not shown in FIG. 4). They may preferably be introduced polyaxially ±20° or more and blocked in an angle-stable manner with the pubic symphysis bone plate 100, as explained herein.

The creeping screws 120 may be made of a titanium alloy or preferably comprise such a titanium alloy. They may have different lengths, preferably between 40 mm and 200 mm.

Furthermore, the creeping screws 120 are cannulated, i.e. they comprise a through-bore along their longitudinal extension. Preferably, this through-bore has an internal diameter of 1.5 mm and up to 3.2 mm.

In certain embodiments, a viscous material (for example bone cement) may be introduced into the bone by the cannulation and transverse bores optionally provided for this purpose.

The embodiments A to F each comprise a head end 121 (bottom in the figure) and a free end 123 (top in the figure) lying opposite the head end 121. The head end 121 is preferably configured at its front/end side to receive a tool, preferably a screwdriver, or a section thereof. The differences between the individual embodiments are described in more detail hereafter.

Embodiment A shows the creeping screw 120, comprising a second external thread 127 (also: partial thread) at its free end 123. The second external thread 127 may be self-tapping or self-drilling in this embodiment as well as in any other embodiment.

Since there is no first external thread 125, the thread-free section 129 extends to the head of the creeping screw 120.

Embodiment B shows the creeping screw 120 with a so-called full thread, i.e. the first external thread 125 and the second external thread 127 merge into each other. The thread preferably extends here-more or less-over the entire length of the creeping screw 120. The first external thread 125 does not comprise a larger external diameter than the second external thread 127 at the free end of the creeping screw 120. The first external thread 125 may optionally also have a larger external diameter than the second external thread 127 at the free end of the creeping screw 120.

Embodiment C substantially corresponds to embodiment A, but a further thread-free section 129′ may be seen at the head end 121, which comprises a larger outer diameter than the comparatively longer thread-free section 129. In some embodiments, the thread-free section 129′ serves to support the creeping screw 120 on or in the bone, in particular in the pelvic bone B, in particular if the creeping screw 120 comprises only a partial thread (see above).

Embodiment D substantially corresponds to embodiment C, but instead of the further thread-free section 129′, a first external thread 125 may be seen at the head end 121, which comprises both a larger outer diameter than the thread-free section 129 and a larger outer diameter than the second external thread 127 at the free end of the creeping screw 120. In some embodiments, the first external thread 125 also serves to support the creeping screw 120 on or in the bone.

Embodiment E substantially corresponds to embodiment D, but a first external thread 125 may be seen at the head end 121, which comprises no larger external diameter than the thread-free section 129 and also no larger external diameter than the second external thread 127 at the free end of the creeping screw 120. In several embodiments, the first external thread 125 also serves to support the creeping screw 120 on or in the bone.

The first external threads 125 of embodiments D and E of FIG. 4 are optionally not tapered along their longitudinal axis and are also not arranged on the head itself. In other embodiments, see embodiment F, this may be provided.

Embodiment F has an external thread on the head itself, which may preferably block itself multidirectionally in the pubic symphysis bone plate 100 (not shown here).

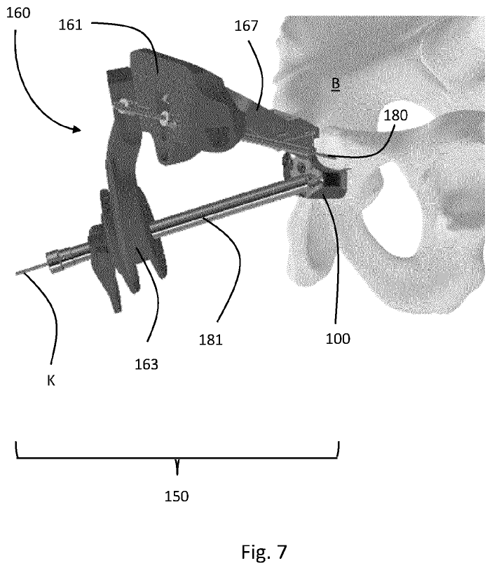

FIG. 5 shows an embodiment of an interlocking screw 130, as may be encompassed in the pubic symphysis bone plate 100 according to the present invention or the set 150 according to the present invention (see e.g. FIG. 7).

Interlocking screws 130 are used to interlock the pubic symphysis bone plate 100 (not shown in FIG. 5) in the (medial) region of the symphysis. They may be inserted mono- or/and polyaxially and are preferably blocked with the bone plate at a stable angle.

The interlocking screw 130 is preferably made of or comprises a titanium alloy or steel alloy.

The interlocking screw 130 may have different lengths, preferably between 10 mm and 50 mm.

The interlocking screw 130 optionally comprises, e.g. at its free end 133, a, preferably self-tapping and/or self-drilling, fourth external thread 137. In the example of FIG. 5, the fourth external thread 137 is a full thread (Ø e.g. between 3 mm and 5 mm), which preferably extends over the entire length of the free end 133.

The interlocking screw 130 further comprises a head end 131, which in turn comprises an engagement on its front/end side, which is designed to receive tools, in particular screwdrivers, or parts thereof.

In the present example of FIG. 5, the head end 131 comprises a third external thread 135 optionally on the head itself, which, due to its design, may serve to block the interlocking screw 130 in the pubic symphysis bone plate 100 with all the properties and advantages mentioned herein. Here, the third external thread 135 preferably has at least a larger external diameter than the fourth external thread 137. FIG. 5 thus shows a tapered external thread.

FIGS. 6a to 6e show a target instrument 160 as part of a set 150 according to the present invention. The target instrument 160 is preferably completely or predominantly radiolucent. It may be made of plastic, for example carbon fiber reinforced PEEK (CFRP), PEEK, POM, etc., anodized aluminum, anodized titanium alloy and/or stainless steel, or may comprise these materials in any technically possible combination.

As set forth herein, in any embodiment, the target instrument 160 may comprise a radiopaque marker, not shown. This may comprise or consist of a circular or annular section.

The target instrument 160 may comprise several or some particular components disclosed herein, and in particular hereinafter, such as, e.g., the components 161, 163, 165, 167, in any combination.

FIG. 6a shows a plate holder 167 configured to releasably connect its lower end to the pubic symphysis bone plate 100 (not shown in FIG. 6a).

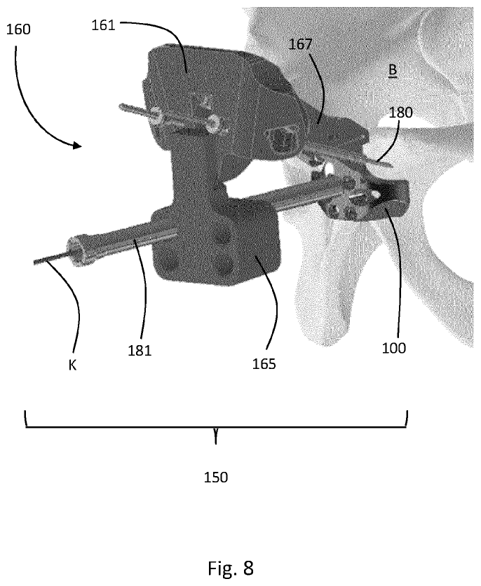

The plate holder 167 may be designed laterally with at least one through-opening 169 (two in this example) for passing a K-wire K therethrough. The through-openings 169 may be simple bores (Ø 1.6 mm to 2.5 mm, preferably Ø 2 mm) for receiving K-wires. The K-wires or sleeve arrangements are used in particular for temporary fastening of the pubic symphysis bone plate 100 together with the target instrument 160 to the pelvic bone (see FIG. 7 and FIG. 8).

The plate holder 167 may be designed to be universal, i.e. suitable for different bone plates, so that it is also suitable, e.g., for connecting it to a humerus plate.

FIG. 6b shows a target bow 161, which is specifically adapted to the pubic symphysis bone plate 100.

In particular, the target bow 161 comprises a plurality of through-openings. Some of these through-openings serve to receive a sleeve arrangement by which K-wires may be inserted into the pelvic bone B for temporary fixation of the target instrument 160 (and thus indirectly of the pubic symphysis bone plate 100), as described in more detail below. In use (see FIG. 6e, FIG. 7 and FIG. 8), other through-openings of the target bow 161 serve to receive, in particular releasably, for example a positioning handle 170 (see FIG. 6e), a guiding block 165 (see FIG. 6c, FIG. 6e and FIG. 8) or a joystick 163 (see FIG. 6d and FIG. 7).

The target bow 161 may have through-openings for passing through a K-wire K or at least one sleeve or combinations thereof. For this purpose, the through-openings optionally have a larger diameter (Ø 5 mm to 12 mm) than the through-openings 169 for K-wires of the plate holder 167.

The K-wire, which may be passed through the target bow 161 and/or the plate holder 167 and inserted into the bone, may serve to temporarily fasten or secure the target bow 160 and/or the pubic symphysis bone plate 100 to the pelvis.

FIG. 6c shows a guiding block 165. The guiding block 165 serves as a monoaxial target device for bone screws, i.e. for fixing the pubic symphysis bone plate 100 to the bone with bone screws, for example interlocking screws 130.

For this purpose, it may be, or may become, connected, preferably releasably, to the target bow 161 (see FIG. 6b). The example of a plug-in connection between target bow 161 and guiding block 165 is shown in FIG. 6e.

FIG. 6d shows a component of the target instrument 160 referred to herein as a joystick 163. The joystick 163 serves as a polyaxial target device for bone screws such as the creeping screws 120 mentioned herein, e.g. for fixing the pubic symphysis bone plate 100 to the bone using bone screws. The polyaxial target device as disclosed herein preferably allows adjustment of the bone screw within the joystick 163 or other component designed for this purpose within an angular range of, e.g., ±20° or more.

For this purpose, the joystick 163 may be, or may become, connected, preferably releasably, to the target bow 161 (see FIG. 6b).

The component referred to here as joystick 163 is exemplarily designed here as a section of a spherical surface with e.g. a circular opening or comprises such an opening. Instead of a spherical surface, the shape of the joystick 163 may also comprise a differently shaped, only optionally curved or straight surface.

The, for example circular, opening in a mostly central or middle area of the joystick 163 is provided for guiding the creeping screw 120, alternatively a sleeve and an instrument which serves to insert the creeping screws 120 or prepares their insertion (for example drill, K-wire or the like). The longitudinal axis of the creeping screw 120 or the sleeve is perpendicular to the surface of the spherical surface. The extension of the longitudinal axis of the creeping screw 120 or the sleeve extends through the center of the desired bore or opening in the pelvic bone B (see FIG. 7 and FIG. 8).

The joystick 163 is optionally shell-shaped with annular concentrically arranged grooves, flutes or elevations around the central guiding of the creeping screw 120 or the sleeve.

FIG. 6e shows the pubic symphysis bone plate 100 together with the target instrument 160, here the plate holder 167 (see FIG. 6a) and the target bow 161 (see FIG. 6b) together with a positioning handle 170.

The positioning handle 170 is used by the user to hold the target instrument before and during temporary fastening to the pelvic bone, in particular during minimally invasive procedures. It is preferably designed radiolucent. It enables the surgeon's hand to be kept out of the radiation field.

The positioning handle 170 may be connected to the target bow 161, in particular releasably, for example by a plug-in connection, as shown in FIG. 6e.

FIG. 6e further illustrates that connecting devices may preferably be used for connecting, which may also be used, for example, for further parts of the target instrument 160, here the guiding block 165. In this way, the openings of the target bow 161, which served to receive the positioning handle 170 before the target instrument was (temporarily) fastened to the pelvic bone, may subsequently serve to receive the guiding block 165 or the joystick 163.

FIG. 7 shows the pubic symphysis bone plate 100 according to the present invention of an embodiment according to the present invention together with a target instrument 160 of an embodiment in a common use. FIG. 7 thus shows an exemplary embodiment of a set 150 according to the present invention.

In the example of FIG. 7, the pubic symphysis bone plate 100 is connected, preferably releasably, to the plate holder 167 of FIG. 6a, for example screwed.

The plate holder 167 is in turn connected to the target bow 161 of FIG. 6b, preferably releasably, for example using a screw.

Two sleeves or through-openings are arranged in the target bow 161, by which sleeves or openings at least one (temporary) fastening device 180, for example a stepped K-wire or as the case here a screw, may be positioned in order to advantageously establish a preferably temporary connection between the target instrument 160 and the pelvic bone B.

Preferably, at least two fastening devices 180 are positioned to the left and right of the symphysis in order to fix the target instrument 160 temporarily but securely to the pelvic bone B.

Alternatively or in addition (e.g., earlier in time), this connection may be made temporarily using stepped K-wires, which may be passed through the target bow 161 and/or through the plate holder 167. The stepped K-wires ensure that they are only inserted into the bone by a controlled length or depth, which in turn helps to avoid damaging surrounding vulnerable structures, such as e.g. the bladder.

The joystick 163 of FIG. 6d is arranged on the target bow 161, preferably releasably connected, for example plugged in. A sleeve 181 was inserted into the center of the joystick 163.

The sleeve 181 may receive a wide variety of instruments and devices, in particular a drill or a K-wire, as shown in FIG. 7. When the drilling is completed, the sleeve 181 may be removed from the joystick 163 and instead a creeping screw 120 (not shown in FIG. 7, see FIG. 4) may be guided through the opening of the joystick and inserted into the bone at a precise angle, in particular along a previously positioned K-wire, screwed into the bone and then blocked.

FIG. 8 shows the pubic symphysis bone plate 100 with parts of the target instrument 160 in a further, common use and thus also the set 150 in a further embodiment.

In the example of FIG. 8, the pubic symphysis bone plate 100 is connected, preferably releasably, to the plate holder 167 of FIG. 6a, for example screwed. The plate holder 167 is in turn connected, in particular releasably, to the target bow 161, e.g. of FIG. 6b, for example screwed, corresponding to FIG. 7.

The fastening device 180 is also arranged according to FIG. 7 for the temporary connection of target instrument 160 and pelvic bone B. To avoid repetition, reference is made to the description with regard to FIG. 7.

The guiding block 165, e.g. of FIG. 6c, is arranged on the target bow 161, preferably releasably connected, for example plugged in. A sleeve 181 is inserted into one of the through-openings of the guiding block 165. This sleeve 181 may receive a wide variety of instruments and devices, in particular a drill or a K-wire, as shown in FIG. 8. When the drilling is completed, an interlocking screw 130 (not shown in FIG. 8, see FIG. 5) may be guided through the lumen of the sleeve 181 by suitable instruments and inserted into the bone at a precise angle, in particular along a previously positioned K-wire, where it is screwed and blocked in order to interlock the pubic symphysis bone plate 100 medially.

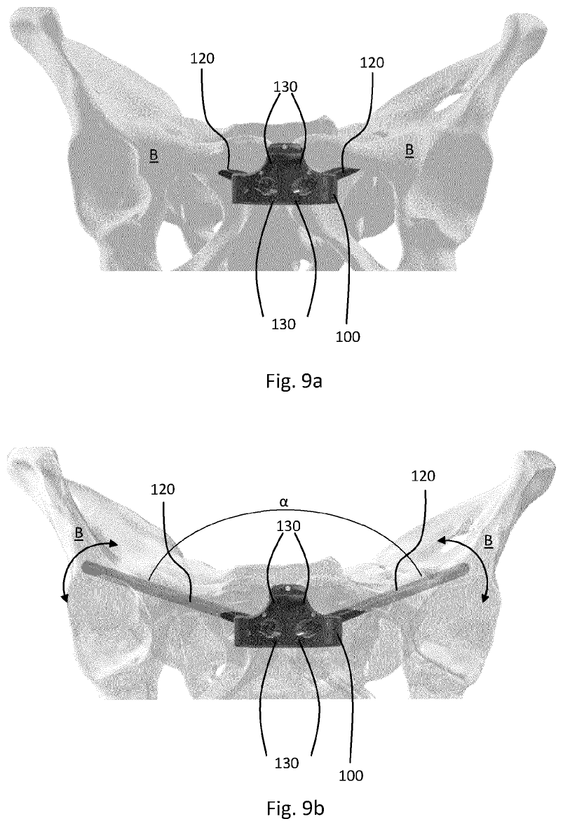

FIG. 9a shows a final osteosynthesis with a view from the front towards the pubic symphysis bone plate 100 according to the present invention.

The pubic symphysis bone plate 100 is screwed to the pelvic bone B with two interlocking screws 130 each on the left and right of the symphysis, respectively.

The creeping screws 120 lie at an opening angle α (see FIG. 9b) to each other within the iliac crest and can thus ensure a stable connection of the pelvic ring.

FIG. 9b shows the final osteosynthesis of FIG. 9a with transparent pelvic bones B in order to illustrate the position of the creeping screws 120 within the pelvic bones B. Reference is made here to the description with regard to FIG. 9a.

Two double arrows are used to show that the angle of the creeping screws 120, which corresponds to the opening angle α, may be aligned to the individual surgical situation.

### LIST OF REFERENCE NUMERALS

100 pubic symphysis bone plate
110k through-opening for creeping screws
110v through-opening for interlocking screws
110z through-opening for the releasable fastening of the target instrument
113 recess
115 compression plate
117 opening for the screw of the compression plate
119 compression screws
120 creeping screw; cannulated screw
121 head end of the creeping screw
123 free end of the creeping screw
125 first external thread
127 second external thread
129 thread-free section of the creeping screw
129′ further thread-free section of the creeping screw
130 interlocking screw
131 top end of the interlocking screw
133 free end of the interlocking screw
135 third external thread
137 fourth external thread
150 set
160 target instrument
161 target bow
163 joystick
165 guiding block
167 plate holder
169 through-opening
170 positioning handle
180 fastening device (temporary)
181 sleeve
B pelvic bone
D1 first diameter
D2 second diameter
K K-wire
R edge
Rₗ left section of the edge
Rₘ middle section of the edge R
Rᵣ right section of the edge
α opening angle

1. A pubic symphysis bone plate for the surgical treatment of damages, in particular fractures or breaks, of the pelvis in the region of the symphysis or the anterior pelvic ring, e.g. up to the acetabulum, preferably including non-displaced or suitably set or re-positioned acetabular fracture, said symphysis bone plate comprising
a plurality of through-openings.

2. The pubic symphysis bone plate according to claim 1, wherein at least one of the through-openings comprises at least two diameters different from each other and/or comprises a step within its inner lumen.

3. The pubic symphysis bone plate according to claim 1, wherein at least one of the through-openings comprises an internal thread.

4. The pubic symphysis bone plate according to claim 1, wherein an upper edge comprises a middle section which extends higher in a caudal-cranial direction than sections bounding it left and right.

5. The pubic symphysis bone plate according to claim 1, comprising a recess for receiving at least a part of the depth of a compression plate.

6. The pubic symphysis bone plate according to claim 1, comprising at least one opening with an internal thread for screwing in a compression screw.

7. A set comprising
a pubic symphysis bone plate according to claim 1;
wherein the set further comprises
at least one cannulated screw or creeping screw, preferably made of titanium alloy, preferably anodized; and/or
at least one interlocking screw and/or
at least one target instrument.

8. The set according to claim 7, wherein at least one of the creeping screws comprises a head end and a free end lying opposite the head end, wherein the creeping screw has a first external thread in the region of its head end and a second external thread in the region of its free end, wherein the first external thread has a larger external diameter than the second external thread.

9. The set according to claim 7, wherein between the first external thread and the second external thread there is arranged the section that is thread-free, at least on its outer side.

10. The set according to claim 7, wherein the head end of the at least one creeping screw comprises a section with an outer diameter which is larger than the outer diameter of a section of the free end lying opposite the head end and/or larger than an outer diameter of the thread-free section.

11. The set according to claim 7, wherein the head end of the at least one creeping screw comprises itself an external thread section at its head.

12. The set according to claim 7, wherein at least one of the interlocking screws comprises a head end and a free end lying opposite the head end, wherein the interlocking screw has a third external thread in the region of its head end and a fourth external thread in the region of its free end wherein the third external thread has a larger external diameter than the fourth external thread.

13. The set according to claim 7, wherein the target instrument comprises:
a target bow; and/or
a joystick; and/or
a guiding block.

14. The set according to claim 7, wherein the target instrument comprises:
a plate holder configured to be releasably connected to the pubic symphysis bone plate.

15. The set according to claim 14, wherein the plate holder comprises at least one through-opening for passing or advancing a K-wire therethrough.

16. The set according to claim 7, comprising a
compression plate;
compression screws for screwing the compression plate against a surface of the pubic symphysis bone plate, preferably in the recess for receiving at least a part of the depth of a compression plate.

17. The set according to claim 7, comprising a
positioning handle, in particular prepared for being releasably connected to the target bow.

18. The set according to claim 7, wherein the target instrument comprises a radiopaque marker and/or a connection point for connecting said target instrument to such a marker.
